(19) United States
(12) Patent Application Publication
(10) Pub. No.: US 2021/0113633 A1
(43) Pub. Date: Apr. 22, 2021

(54) PROBIOTIC COMPOSITIONS AND USES THEREOF

(71) Applicant: Probi AB, Lund (SE)

(72) Inventors: Claes Sven Anders Ohlsson, Göteborg (SE); Niklas Larsson, Lund (SE); Irini Lazou Ahrén, Lund (SE)

(21) Appl. No.: 17/252,863

(22) PCT Filed: Jun. 13, 2019
(86) PCT No.: PCT/EP2019/065588

§ 371 Date: Dec. 16, 2020

(30) Foreign Application Priority Data
Jun. 18, 2018 (GB) 1809947.3
Apr. 16, 2019 (GB) 1905389.1

(51) Int. Cl. A61K 35/747; A61P 19/10; A61K 31/593

(52) U.S. Cl. CPC A61K 35/747; A61K 31/593; A61P 19/10

(57) ABSTRACT

The invention relates to at least one probiotic strain chosen from Lactobacillus paracasei 8700:2 (DSM 13434) and/or at least one probiotic strain of Lactobacillus piantarum, for use in the treatment and/or prevention of trabecular bone loss, in a mammal, preferably in anon-rodent mammal, more preferably in a human, most preferably in a peri-menopausal woman, post-menopausal woman or a woman six years or less after onset of menopause.

### TECHNICAL FIELD OF THE INVENTION

The present invention relates to at least one probiotic strain chosen from Lactobacillus paracasei 8700:2 (DSM 13434) and/or at least one probiotic strain of Lactobacillus plantarum, for use in the treatment and/or prevention of trabecular bone loss in a mammal. Preferably, the use is for treating a non-rodent mammal, more preferably a human, and most preferably a peri-menopausal woman, post-menopausal woman, or a woman six years or less after onset of menopause.

### BACKGROUND OF THE INVENTION

Bone tissue is a mineralized tissue of two types, cortical (also known as compact) bone and trabecular (also known as cancellous or spongy) bone.

Cortical bone is denser and stronger than trabecular bone and forms the hard exterior (cortex) of bones. Microscopically, cortical bone in humans is composed of osteons, columns formed of concentric rings of calcified matrix called lamellae that surround a central (Haversian) canal containing the blood vessels, nerves and lymphatic vessels. Cortical bone typically has an outer surface of periosteum connective tissue and an inner surface of endosteum connective tissue that forms the boundary between cortical and trabecular bone.

Trabecular bone is the internal tissue of the skeletal bone and is an open cell porous network comprising tiny lattice-shaped units (trabeculae). Trabecular bone has a higher surface-area-to-volume ratio than cortical bone because it is less dense, making it softer and weaker, but more flexible. The greater surface area also makes it suitable for metabolic activities such as the exchange of calcium ions. Trabecular bone is typically found at the ends of long bones, near to joints and within the interior of vertebrae. Microscopically, the primary anatomical and functional unit of trabecular bone is the trabecula. Unlike the concentric circles of the osteons, trabeculae typically form an irregular network of thin rod-like formations of osteoblasts covered in endosteum, and the spaces between are filled with bone marrow and hematopoietic stem cells. Trabecular bone accounts for approximately 20% of total bone mass but has nearly ten times the surface area compared to cortical bone.

Other types of tissue found in bones include bone marrow, endosteum, periosteum, nerves, blood vessels and cartilage.

There are five types of bones in the human (mammalian) body, characterised by their shape: long, short, flat, irregular, and sesamoid. ‘Long bones’, e.g. the femur and most other bones of the limbs, fingers and toes, typically have a shaft (diaphysis) mostly made of cortical bone that forms a cavity filled with lesser amounts of marrow, and a rounded head (epiphysis) at each end of the bone. An epiphysis typically comprises trabecular bone surrounded by layers of cortical bone. ‘Short bones’, e.g. those of the wrist and ankle, are roughly cube-shaped and typically comprise a thin layer of cortical bone surrounding an interior of trabecular bone. ‘Flat bones’, e.g. the sternum, ribs, hips and most of the skull bones, are thin and generally curved, with two parallel layers of cortical bone sandwiching a layer of trabecular bone. ‘Irregular bones’, e.g. vertebrae, sacrum, coccyx, temporal, sphenoid, ethmoid, zygomatic, maxilla, mandible, palatine, inferior nasal concha, and hyoid, have an irregular shape and typically comprise thin layers of compact bone surrounding an interior of trabecular bone. Sesamoid bones, e.g. patella and pisiform, are bones embedded in tendons.

Bone tissue (osseus tissue) of both cortical and trabecular bone typically comprises a relatively small number of cells trapped in a tough matrix of collagen (ossein) fibres on which inorganic salt crystals (e.g. calcium hydroxylapatite/hydroxyapatite) adheres to strengthen the bone. The remainder of the matrix is typically filled with ground substance—an amorphous gel-like substance in the extracellular space that contains all components of the extracellular matrix including water, glycosaminoglycans (GAGs; e.g. hyaluronan), proteoglycans which GAGs are bound to (e.g. heparan sulfate and keratin sulfate), glycoproteins (e.g. osteonectin, osteopontin, bone sialoprotein), osteocalcin, and link proteins (e.g. vinculin, spectrin and actomysin). Bone is formed by the hardening of the matrix around entrapped cells, which typically change from osteoblasts to inactive osteocytes.

Osteoporosis is a disease in which bones become fragile and more likely to fracture. Usually the bone loses density, which measures the amount of calcium and minerals in the bone. Osteoporosis is the most common type of bone disease. About half of all women over the age of 50 will have a fracture of the hip, wrist, or vertebra (bone of the spine) during their lifetime. Bone is living tissue. Existing bone is constantly being replaced by new bone. Osteoporosis occurs when the body fails to form enough new bone, when too much existing bone is reabsorbed by the body, or both. Calcium is one of the important minerals needed for bones to form. If you do not get enough calcium and vitamin D, or your body does not absorb enough calcium from your diet, your bones may become brittle and more likely to fracture. A drop in estrogen in women at the time of menopause and a drop in testosterone in men is a leading cause of bone loss.

Fractures caused by osteoporosis constitute a major health concern and result in a huge economic burden on health care systems. The lifetime risk of any osteoporotic fracture is high in the western world (around 50% for women and 20% for men) and fractures are associated with significant mortality and morbidity. Cortical bone constitutes approximately 80% of the bone in the body and several studies have shown that cortical bone is the major determinant of bone strength and thereby fracture susceptibility. Bone loss after the age of 65 is mainly due to loss in cortical bone and not trabecular bone (Lancet, 2010, May 15; 375(9727): 1729-36). Nevertheless, the risk of fracture is greater at skeletal sites where trabecular bone is predominant, particularly the head of the femur, the vertebrae and the distal radius, which are the most common fracture sites

(McDonnell et al 2007, Ann Biomed Eng, 35(2): 170-189). Further, patients who have already had a vertebral fracture are more likely to experience further fractures within one year and this likelihood increases with the number of fractures sustained (McDonnell et al 2007, supra).

The skeleton is remodeled by bone forming osteoblasts (OBs) and bone resorbing osteoclasts (OCLs). Macrophage colony stimulating factor (M-CSF) increases proliferation and survival of OCLs precursor cells as well as up-regulates expression of receptor activator of nuclear factor-KB (RANK) in OCL. This allows RANK ligand (RANKL) to bind and start the signalling cascade that leads to OCL formation. The effect of RANKL can be inhibited by Osteoprotegerin (OPG), which is a decoy receptor for RANKL.

Osteoporotic bone loss occurs due to an imbalance in the remodelling process. This may occur by a combination of increased resorption activity, with deeper cavities being formed by the osteoclasts, and insufficient formation of replacement bone tissue by the osteoblasts. Remodelling activity is low in the peripheral skeleton and high in the central skeleton, and this increases the risk of fractures due to bone loss in the vertebrae (McDonnell et al 2007, supra).

The association between inflammation and bone loss is well established and in auto-immune diseases osteoclastic bone resorption is driven by inflammatory cytokines produced by activated T-cells. In addition, several studies demonstrate that low-grade systemic inflammation, indicated by moderately elevated serum levels of high sensitivity C-reactive protein (hsCRP), associate with low BMD, elevated bone resorption and increased fracture risk. The estrogen deficiency that occurs after menopause results in increased formation and prolonged survival of osteoclasts. This is suggested to be due to a number of factors including loss of the immunosuppressive effects of estrogen, resulting in increased production of cytokines promoting osteoclastogenesis, and direct effects of estrogen on OCLs. In line with these data, blockade of the inflammatory cytokines TNFα and IL-1 leads to a decrease in bone resorption markers in early postmenopausal women.

In recent years, the importance of the gut microbiota (GM) for both health and disease has been intensively studied. The GM consists of trillions of bacteria which collectively contain 150-fold more genes than our human genome. It is acquired at birth and, although a distinct entity, it has clearly coevolved with the human genome and can be considered a multicellular organ that communicates with and affects its host in numerous ways. The composition of the GM is modulated by a number of environmental factors such as diet and antibiotic treatments. Molecules produced by the gut bacteria can be both beneficial and harmful and are known to affect endocrine cells in the gut, the enteric nervous system, gut permeability and the immune system. Perturbed microbial composition has been postulated to be involved in a range of inflammatory conditions, within and outside the gut including Crohn's disease, ulcerative colitis, rheumatoid arthritis, multiple sclerosis, diabetes, food allergies, eczema and asthma as well as obesity and the metabolic syndrome.

Probiotic bacteria are defined as live microorganisms which, when administered in adequate amounts, confer a health benefit on the host and are believed to alter the composition of the gut microbiota.

WO 2014/163568 discloses experimental data reporting administration of the probiotic strain Lactobacillus paracasei 8700:2 (DSM 13434) and the combination of the probiotic strains Lactobacillus paracasei 8700:2 (DSM 13434), Lactobacillus plantarum HEAL 9 (DSM 15312) and Lactobacillus plantarum HEAL 19 (DSM 15313) to ovariectomized (ovx) mice as a model of osteoporosis, particularly post-menopausal osteoporosis. Significant protective effects were reported on cortical bone in ovx mice but not on trabecular bone in ovx mice.

Thus, there is still a need within the art to find effective preventive and therapeutic methods against trabecular bone loss in a mammal, and particularly in humans.

### DESCRIPTION OF THE INVENTION

A first aspect of the invention provides at least one probiotic strain chosen from Lactobacillus paracasei 8700:2 (DSM 13434) and/or at least one probiotic strain of Lactobacillus plantarum, for use in the treatment and/or prevention of trabecular bone loss in a mammal.

The use according to the first aspect of the invention may be by increasing the absorption of Ca²⁺ ions in a mammal.

By “use in the treatment and/or prevention” we include the meaning of a use which gives rise to an effect in a subject of preventing, delaying, protecting against, reducing the severity of and/or removing, one or more symptoms and/or other markers associated with a disease or condition.

By “treat”, “treatment” or “treating” we include the meaning that the event or condition being treated is ameliorated, reduced in severity, removed, blocked from occurring further, protected against occurring further, delayed and/or made to cease. Such treatment typically takes place after the event (or the same kind of event) has occurred or the condition is manifest. It will also be appreciated that such terms may include the meaning that an event or condition is maintained in the current state without becoming worse or developing further.

By “prevent”, “prevention” or “preventing” we include the meaning that the event or condition being prevented is protected against, delayed, reduced (e.g. reduced in severity), blocked from occurring, or made to cease. Such prevention typically takes place before the event occurs or the condition is manifest, but it will be appreciated that it can also mean to prevent further occurrence of the same kind of event. It will also be appreciated that such terms may include the meaning that an event or condition is maintained in the current state without becoming worse or developing further.

For example, a measure of trabecular bone loss (e.g. trabecular bone mineral content, trabecular bone mineral density) following administration of the at least one probiotic strain (or of a composition comprising the at least one probiotic strain) according to the first aspect of the invention may be reduced by at least 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98% or at least 99% compared to without administration of the at least one probiotic strain, or compared to administration of a corresponding composition lacking the at least one probiotic strain. A minimum of one region of trabecular bone should be measured in the mammal. For example, it is preferred that the trabecular bone loss is measured at the lumbar spine. The trabecular bone loss may also be measured at the knee joint, for example by measuring bone volume and/or bone thickness of the epiphyseal trabecular bone (including subchondral bone), such as epiphyseal trabecular bone of the tibia or femur at the knee joint (see also, for example, Milz and Putz, 1994, Quantitative morphology of the subchondral plate of the tibial plateau, J Anat 185(Pt 1):103-110, the entire contents of which are incorporated herein by reference).

By “trabecular bone loss” we include the meaning that bone mineral content and/or bone mineral density of trabecular bone is reduced over time.

Preferably, the at least one probiotic strain for use according to the first aspect of the invention is effective to treat and/or prevent trabecular bone loss at the lumbar spine.

Preferably, the at least one probiotic strain for use according to the first aspect of the invention is effective to treat and/or prevent trabecular bone loss at the knee joint, particularly one or both of tibial epiphyseal trabecular bone (trabecular bone at the proximal epiphysis of the tibia) and femoral epiphyseal trabecular bone (trabecular bone at the distal epiphysis of the femur, such as part of one or both of the femoral condyles).

The upper part of the tibia is called the proximal tibia or proximal tibial epiphysis. According to McKinnis (2014, Fundamentals of Musculoskeletal Imaging, 4^{th} ed, F. A. Davis Company, Philadelphia; particularly FIG. 13-1, accessible on 16 Apr. 2019 at https://fadavispt.mhmedical.com/content.aspx?bookid=1899&sectionid=141191793) the proximal tibia consists of medial and lateral condyles, which superiorly form the articular surface of the tibia, or the tibial plateau. Similarly, the lower part of the femur is called the distal femur or distal femoral epiphysis. According to McKinnis (2014, supra), the distal femur exhibits medial and lateral condyles (see FIG. 13-1 of McKinnis, supra).

By “subchondral” we include the meaning of the layer of bone just below the cartilage in a joint. Hence, in the knee joint, the subchondral bone of the proximal tibia is just below (distal to) the cartilage of the knee joint, and the subchondral bone of the distal femur is just ‘below’ (proximal to) the cartilage of the knee joint. Subchondral bone consists of a cortical part (subchondral bone plates) and a trabecular part (subchondral trabecular bone), for example, see FIG. 1 of Arijmand et a! (2019, Sci Rep 8(1):11478).

By “epiphyseal” we include the meaning of the region of the bone comprising the epiphysis, including subchondral bone. Hence, for bones of the knee joint, e.g. the tibia, “epiphyseal bone” includes the regions named as subchondral and epiphyseal in FIG. 1 of Arijmand et al, supra.

By “bone mineral content” (BMC) we include the meaning of the amount of bone minerals (e.g. calcium) in bone tissue. Measures of BMC typically include g and g/cm. Preferably BMC is measured in grams (g).

By “bone mineral density” (BMD) we include the meaning of the amount of bone minerals (e.g. calcium) in bone tissue, expressed as a density. Measures of BMD typically include mass of mineral per volume of bone (e.g. cubic centimetre) or, when assessed by clinical imaging (densitometry), optical density per area (e.g. square centimetre) of bone surface. For example, BMD may be expressed in g/cm² or in g/cm³. Hence, BMD may be calculated from BMC in g by dividing by the surface area of the bone, or from BMC in g/cm by dividing by the width of the bone at the scanned line. BMD is typically a measure of the strength of a bone, and reduced bone mineral density may increase the chance of fractures, osteopenia and osteoporosis.

Bone mineral content and bone mineral density may be measured by any method known in the art. For example, BMC and BMD may be measured by dual-energy X-ray absorptiometry (DXA or DEXA), dual X-ray absorptiometry and laser (DXL), quantitative computed tomography (QCT), quantitative ultrasound (QUS), single photon absorptiometry (SPA), dual photon absorptiometry (DPA), digital X-ray radiogrammetry (DXR) or single energy X-ray absorptiometry (SEXA). DXA is the most widely used technique but QCT (or X-ray micro-computed tomography [micro-CT] for small animals) is preferred as it is capable of measuring the bone's volume.

Trabecular bone loss and/or trabecular bone mineral content can be measured by any suitable method known in the art for measuring BMD and/or BMC in trabecular bone, including those listed above.

The use according to the first aspect of the invention may comprise the treatment and/or prevention of trabecular bone loss associated with osteopenia.

Osteopenia is a condition in which bone mineral density is lower than normal. It is considered by many doctors to be a precursor to osteoporosis. However, not every person diagnosed with osteopenia will develop osteoporosis. Preferably, osteopenia is defined as having a bone mineral density T-score (e.g. at total hip and/or spine such as lumbar spine) of −1>T>−2.5.

Bone mineral density T-score (or ‘T-score’ as used herein) typically represents the number of standard deviations above or below the mean for the bone mineral density of a healthy reference 30-year-old adult. For example the healthy reference 30-year-old adult may be of the same sex and ethnicity as the patient, or may be a white female. A T-score of −1 or higher is indicative of normal (healthy) bone density and a T-score of −2.5 or lower is indicative of osteoporosis. Preferably bone mineral density and/or bone mineral density T-score are measured at the lumbar spine, but they may also be measured in other skeletal regions e.g. another spinal region, total hip, femoral neck, knee joint, tibial epiphyseal trabecular bone (including subchondral bone), femoral epiphyseal trabecular bone (including subchondral bone). It will be appreciated that bone mineral density T-score can be calculated using a bone mineral density measurement made by any suitable method known in the art, including the methods described above. Preferably bone mineral density T-score is calculated using a bone mineral density measurement obtained by dual energy X-ray absorptiometry (DXA).

Patient Group

The at least one probiotic strain according to the first aspect of the invention is suitable to be used in mammals. The at least one probiotic strain according to the first aspect of the invention may be suitable for rodent mammals, e.g. mice, rats, guinea pigs. Preferably, the at least one probiotic strain is suitable to be used by non-rodent mammals, e.g. cats, dogs, horses, monkeys.

Most preferably, the at least one probiotic strain is suitable for humans (men and/or women), including elderly people (such as humans older than 50, 55, 60, 65, 70, 75, 80, 85, 90, or 95 years), post-menopausal women, peri-menopausal women and pre-menopausal women, as these are individuals in which trabecular bone loss and trabecular bone mineral content loss are or may typically become a problem.

Otherwise healthy people may also benefit from the invention in order to prevent getting trabecular bone loss, which can lead to osteoporosis.

Most preferably, the at least one probiotic strain is suitable for use by a post-menopausal woman, for example, a woman within one, two, three, four, five, six, seven, eight, nine, ten, eleven or twelve years from onset of menopause, and most preferably a woman six years or less after onset of menopause.

Preferably, the at least one probiotic strain is suitable for use in a woman older than 45, 50, 55 or 60. For example, the at least one probiotic strain may be suitable for use in a woman between the ages of 45 and 65, such as between 45 and 50, 50 and 55, 55 and 60, 60 and 65, 45 and 55, 50 and 60, 55 and 65, 45 and 60, 50 and 65, or 45 and 65.

Menopause is the time in most women's lives when menstrual periods stop permanently, and they are no longer able to bear children. Menopause typically occurs between 49 and 52 years of age. Medical professionals often define menopause as having occurred when a woman has not had any vaginal bleeding for a year. Hence, the date of menopause itself is typically determined retroactively, once 12 months have passed after the last appearance of menstrual blood. At the physiological level, menopause happens because of a decrease in the ovaries' production of the hormones oestrogen and progesterone. Hence, menopause may also be defined by a decrease in the production of these hormones by the ovaries, and a diagnosis of menopause can be confirmed by measuring hormone levels in the blood or urine. In those who have had surgery to remove their uterus but still have ovaries, menopause may be viewed to have occurred at the time of the surgery or when their hormone levels fell. Following the removal of the uterus, symptoms of menopause typically occur earlier.

The term “pre-menopause” includes the meaning of the years leading up to the last menstrual period, when the levels of reproductive hormones are becoming more variable and lower, and the effects of hormone withdrawal are present. Pre-menopause typically starts before the monthly cycles become noticeably irregular in timing. A “pre-menopausal woman” is a woman in her time of pre-menopause.

The term “peri-menopause” (literally ‘around the menopause’) refers to the menopause transition years and is typically a time before and after the date of the final episode of menstrual flow. Hence, a “peri-menopausal woman” is a woman in her time of perk menopause. Typically, peri-menopause begins between 40 and 50 years of age (average 47.5 years) and may last for four to ten years. For example, peri-menopause may be four to eight years, beginning with the time of changes in the length of times between periods and ending one year after the final menstrual period (The North American Menopause Society, https://web.archive.org/web/2013041011 1346/http://www.menopause.org/for-women/menopauseflashes/menopause-101-a-prime r-for-the-perimenopausal). Alternatively, peri-menopause may be six to ten years ending 12 months after the last menstrual period (Dr Jerilynn C. Prior, Centre for Menstrual Cycle and Ovulation Research, https://web.archive.org/web/2013022505 5347/http://cemcor.ca/help_yourself/perimenopause). Oestrogen levels average about 20-30% higher during peri-menopause than during pre-menopause, often with wide fluctuations. These fluctuations cause many of the physical changes during perimenopause as well as menopause, including hot flashes, night sweats, difficulty sleeping, vaginal dryness or atrophy, incontinence, osteoporosis, and heart disease.

The term “postmenopausal” typically describes women who have not experienced any menstrual flow for a minimum of 12 months (thus confirming that menstrual cycles have ceased), assuming that they have a uterus and are not pregnant or lactating. In women without a uterus, menopause or post-menopause can be identified by a blood test showing a very high level of follicle stimulating hormone (FSH). Thus, post-menopause may also be defined as the time after the point when a woman's ovaries become inactive. As a woman's reproductive hormone levels continue to drop and fluctuate for some time into post-menopause, hormone withdrawal effects such as hot flashes may take several years to disappear.

Alternatively, the at least one probiotic strain may be suitable for use by men, such as a man older than 45, 50, 55 or 60, or a man between the ages of 45 and 65, such as between 45 and 50, 50 and 55, 55 and 60, 60 and 65, 45 and 55, 50 and 60, 55 and 65, 45 and 60, 50 and 65, or 45 and 65.

The at least one probiotic strain according to the first aspect of the invention may also be suitable for use in a mammal with osteopenia, such as a human with a bone mineral density T-score of −1>T>−2.5, preferably when T-score is measured at the lumbar spine.

Probiotic Strains

Probiotic bacteria are defined as “live microorganisms that, when administered in adequate amounts, confer a health benefit on the host” (Hill et al, Nat Rev Gastroenterol Hepatol, 2014, 11(8):506-514). Bacteria of the genera Lactobacillus and Bifidobacterium are the most frequently used bacteria in probiotic products. These bacteria are generally safe, as are probiotic products based on these organisms. For a bacterium to fulfil the definition of a probiotic it typically has to be able to survive in and colonise the intestines, survive the processes of production and storage, and have evidence that it has positive effects on consumer health.

By “at least one probiotic strain” we include the meaning of one or more strain(s) of bacteria which when administered in adequate amounts confer a health benefit on the host. Typically, the administration of said at least one probiotic strain will alter the composition of the gut microbiota.

The “at least one probiotic strain” according to the first aspect of the invention may be Lactobacillus paracasei 8700:2 (DSM 13434).

Lactobacillus paracasei 8700:2, DSM 13434, was deposited on 6 April 2000 at DSMZ-DEUTSCHE SAMMLUNG VON MIKROORGANISMEN UND ZELLKULTUREN GmbH, Mascheroder Weg 1b, D-38124 Braunschweig, Germany, by Probi AB.

The “at least one probiotic strain” according to the first aspect of the invention may be at least one probiotic strain of Lactobacillus plantarum.

Preferably, the at least one probiotic strain of Lactobacillus plantarum is chosen from Lactobacillus plantarum 299 (DSM 6595), Lactobacillus plantarum 299v (DSM 9843), Lactobacillus plantarum HEAL 9 (DSM 15312), Lactobacillus plantarum HEAL 19 (DSM 15313), Lactobacillus plantarum HEAL 99 (DSM 15316), Lactobacillus plantarum GOS42 (DSM 32131), Lactobacillus plantarum DSM 17852 (LB3e) and Lactobacillus plantarum DSM 17853 (LB7c).

Lactobacillus plantarum 299 (DSM 6595) was deposited on 2 Jul. 1991 at DSM-DEUTSCHE SAMMLUNG VON MIKROORGANISMEN UND ZELLKULTUREN GmbH, Mascheroder Weg 1 B, D-3300 Braunschweig, Germany, in the name of Probi (i.e. Probi AB).

Lactobacillus plantarum 299v (DSM 9843) was deposited on 16 Mar. 1995 at DSM-DEUTSCHE SAMMLUNG VON MIKROORGANISMEN UND ZELLKULTUREN GmbH, Mascheroder Weg 1b, D-38124 Braunschweig, Germany, by Probi AB.

Lactobacillus plantarum HEAL 9, DSM 15312, Lactobacillus plantarum HEAL 19, DSM 15313, and Lactobacillus plantarum HEAL 99, DSM 15316 were deposited on 27 November 2002 at DSMZ-DEUTSCHE SAMMLUNG VON MIKROORGANISMEN UND ZELLKULTUREN GmbH, Mascheroder Weg 1b, D-38124 Braunschweig, Germany, by Probi AB.

Lactobacillus plantarum GOS42 (DSM 32131) was deposited on 2 Sep. 2015 at Leibniz Institute DSMZ-German Collection of Microorganisms and Cell Cultures, Inhoffenstr. 7 B, D-38124 Braunschweig, Germany by Probi AB.

Lactobacillus plantarum DSM 17852 (LB3e) and Lactobacillus plantarum DSM 17853 (LB7c) were deposited on 6 January 2006 at DSMZ-Deutsche Sammlung von Mikroorganismen and Zellkulturen GmbH, Mascheroder Weg 1b, D-38124 Braunschweig, Germany, by Probac AB. All rights and duties in connection with microorganism deposits DSM 17852 and DSM 17853 were subsequently given to and accepted by Probi AB, who is now the depositor of the DSM 17852 and DSM 17853 strains.

In an embodiment according to the first aspect of the invention, Lactobacillus paracasei 8700:2 (DSM 13434) is intended for use in combination with at least one probiotic strain of Lactobacillus plantarum. For example, Lactobacillus paracasei 8700:2 (DSM 13434) may be used in combination with one, two or more probiotic strains of Lactobacillus plantarum.

In a most preferred embodiment according to the first aspect of the invention, the at least one probiotic strain is Lactobacillus paracasei 8700:2 (DSM 13434) in combination with Lactobacillus plantarum HEAL 9 (DSM 15312) and Lactobacillus plantarum HEAL 19 (DSM 15313).

The probiotic strains according to the first aspect of the invention may be viable, inactivated or dead. Preferably, the probiotic strains are viable. For example, preferably the probiotic strains are freeze-dried.

Compositions

The at least one probiotic strain according to the first aspect of the invention may be present in a composition comprising at least one suitable carrier. For example, the carrier may be a diluent or excipient. The composition may be as a solid or liquid formulation, and hence the at least one carrier may be a solid or a liquid, or may comprise bath at least one solid component and at least one liquid component.

Examples of a suitable liquid carrier include water, milk, coconut water, fruit drinks and juices, milk substitutes (soya drink, oat drink, nut and other plant-based drinks), sparkling beverages, glycerin, propylene glycol and other aqueous solvents.

Examples of a suitable solid carrier or excipient include maltodextrin, inulin, a cellulose such as microcrystalline cellulose (MCC), hydroxypropylmethylcellulose (HPMC) or hydroxy-propylcellulose (HPC), sugar alcohols, high molecular weight polyethylene glycols, lactose, sodium citrate, calcium carbonate, dibasic calcium phosphate and glycine, disintegrants such as starch (preferably corn, potato, tapioca or other vegetable starch), sodium starch glycollate, croscarmellose sodium and certain complex silicates, and granulation binders such as polyvinylpyrrolidone, sucrose, gelatin and acacia. Additionally, lubricating agents such as magnesium stearate, stearic acid, glyceryl behenate and talc may be included.

In an embodiment according to the first aspect of the invention, the carrier may be selected from a pharmaceutically acceptable carrier, a pharmaceutically acceptable excipient, a food-grade carrier, a food-grade excipient, a diluent and a food.

Examples of suitable pharmaceutically acceptable carriers, excipients and diluents include those well known to a skilled person in the art, for example those given in Remington: The Science and Practice of Pharmacy, 19^{th} ed., vol. 1 & 2 (ed. Gennaro, 1995, Mack Publishing Company).

By “food-grade” we include carriers, ingredients and excipients that meet the ‘generally recognized as safe’ (GRAS) criteria.

By “food” we include any substance for consumption to provide nutritional benefit or support for an organism. Examples of suitable food carriers include beverages (e.g. juices), dairy products (e.g. yoghurts, cheese, ice creams, infant formula and spreads such as margarine), dairy-alternative products (e.g. soy, nut or other plant-based drinks, yoghurts and spreads), cereal-based products (e.g. breads, biscuits, breakfast cereals, pasta and dry food bars such as health bars), and baby food (e.g. pureed fruit and/or vegetable).

The composition according to the first aspect of the invention may be a dry, non-fermented composition, a fermented composition, or a dry, fermented composition. Fermentation in this context particularly includes lactic acid fermentation by lactic acid bacteria in anaerobic conditions. In the case of a dry, non-fermented composition, substantially no fermentation takes place before ingestion by a subject, and so fermentation only takes place in the gastrointestinal tract after ingestion of the composition by a subject.

Hence, in some embodiments according to the first aspect of the invention, the composition is in the form of a food wherein the food is a cereal-based product, a dairy product, a juice drink, or a fermented food.

Examples of fermented foods include fermented milk products (such as yoghurt, kefir or lassi), fermented dairy-free milk alternatives (such as coconut milk kefir), fermented cereal-based products (such as oats, oatmeal, maize, sorghum, wheat), fermented vegetables (such as sauerkraut, kimchi, or pickles), fermented legumes or soybeans (such as natto or tempeh) and fermented tea (such as kombucha).

In some embodiments according to the first aspect of the invention, the at least one probiotic strain is present in a composition that is not naturally occurring, e.g. the composition comprises more than the probiotic strain(s) and water.

In use, the at least one probiotic strain or the composition comprising the at least one probiotic strain according to the first aspect of the invention may be mixed with a liquid or solid carrier before administration to a mammal. For example, a subject may mix the at least one probiotic strain or the composition thereof with a carrier comprising one or more liquids chosen from water, milk, coconut water, fruit drinks and juices, milk substitutes (soya drink, oat drink, nut and other plant-based drinks), sparkling beverages or some other aqueous solvent or drink prior to intake. Similarly, the at least one probiotic strain or the composition thereof may be mixed with a carrier consisting of one or more foods.

Suitable food carriers include oatmeal carrier, barley carrier, fermented or non-fermented dairy products such as yoghurts, ice creams, milkshakes, fruit juices, beverages, soups, breads, biscuits, pasta, breakfast cereals, dry food bars including health bars, plant-based foods such as soy products, spreads, baby food, infant nutrition, infant formula, breast milk replacements from birth.

Preferably, the formulation is a unit dosage containing a daily dose or unit, daily sub-dose or an appropriate fraction thereof, of the composition comprising the probiotic strains.

The composition according to the first aspect of the invention may be a dietary supplement. By “dietary supplement” we include the meaning of a manufactured product intended to supplement the diet when taken by mouth, e.g. as a pill, capsule, tablet, or liquid. Dietary supplements may contain substances that are essential to life and/or those that have not been confirmed as being essential to life but may have a beneficial biological effect. When the composition according to the first aspect of the invention is in the form of a dietary supplement the carrier(s) to be added include those well known to a skilled person in the art, for example those given in Remington: The Science and Practice of Pharmacy, 19^{th} ed., vol. 1 & 2 (ed. Gennaro, 1995, Mack Publishing Company). Any other ingredients that are normally used in dietary supplements are known to a skilled person and may also be added conventionally together with the at least one probiotic strain.

The composition according to the first aspect of the invention may be provided in the form of a solution, suspension, emulsion, tablet, granule, powder, capsule, lozenge, chewing gum, or suppository.

For example, in a preferred embodiment, the composition according to the first aspect of the invention is a dietary supplement in the form of a capsule comprising freeze-dried Lactobacillus, such as a dietary supplement in the form of a capsule comprising 10¹⁰ CFU freeze-dried Lactobacillus.

In an embodiment according to the first aspect of the invention, the at least one probiotic strain is present (e.g. in a composition) in an amount from about 1×10⁶ to about 1×10¹⁴ CFU/dose, preferably from about 1×10⁸ to about 1×10¹² CFU/dose, more preferably from about 1×10⁹ to about 1×10¹¹ CFU/dose, and most preferably about 1×10¹⁰ CFU/dose. If the at least one probiotic strain consists of more than one probiotic strain, such amounts represent the total CFU/dose of the combination of probiotic strains. For example, the at least one probiotic strain may be present in an amount from about 1×10⁶, 1×10⁷, 1×10⁸, 1×10⁹, 1×10¹⁰, 1×10¹¹, 1×10¹² or about 1×10¹³ CFU/dose. The at least one probiotic strain may be present in an amount to about 1×10¹⁴, 1×10¹³, 1×10¹², 1×10¹¹, 1×10¹⁰, 1×10⁹, 1×10⁸ or about 1×10⁷ CFU/dose. The at least one probiotic strain according to the first aspect of the invention may also be used alone in water or any other aqueous vehicle in which the at least one probiotic strain is added or mixed before ingestion.

In an embodiment according to the first aspect of the invention the composition is supplemented with vitamin D. For example, the vitamin D may be in the form of vitamin D3 cholecalciferol or vitamin D2 ergocalciferol. Preferably the vitamin D is in the form of vitamin D3 cholecalciferol. The recommended daily intake (RDI) of vitamin D is 400 international units (IU) (approximately 10 μg) for children up to age 12 months, 600 IU (approximately 15 μg) for ages 1 to 70 years, and 800 IU (approximately 20 μg) for people over 70 years (https://ods.od.nih.gov/factsheetsNitaminD-HealthProfessional/), but some health agencies recommend 10 μg (approximately 400 IU) per day or 15 μg (approximately 600 IU) per day. The amount of vitamin D with which the composition may be supplemented may be, for example, up to 10, 20, 30, 40, 50, 60, 70, 80, 90, 100, 200, 300, 400, 500, 600, 700, or 800 IU, or higher, or up to 0.5, 1, 1.5, 2, 2.5, 3, 4, 5, 7.5, 10, 12.5, 15, 17.5 or 20 μg, or higher.

In an embodiment according to the first aspect of the invention the composition is supplemented with Ca²⁺ in the form of for instance a salt, e.g. calcium carbonate, calcium chloride, calcium salts of citric acid, calcium gluconate, calcium glycerophosphate, calcium lactate, calcium oxide, calcium sulphate. The recommended daily intake (RDI) of Ca²⁺ is 800 mg. The amount of Ca²⁺ with which the composition may be supplemented may be, for example, up to 320 mg, 300 mg, 250 mg, 200 mg, 180 mg, 160 mg, 140 mg, 120 mg, 100 mg, 80 mg, 60 mg, 50 mg, 40 mg, 30 mg, 20 mg, or up to 10 mg.

The composition according to the first aspect of the invention can be administered orally, buccally or sublingually in the form of tablets, capsules, powders, ovules, elixirs, solutions or suspensions, which may contain flavouring or colouring agents, for immediate-, delayed- or controlled-release applications. The composition may be administered in the form of a powdered composition such as a fast-melt microbial composition, for example those described in WO 2017/060477, or in Probi's UK Patent Application 1708932.7 or Probi's publication WO 2018/224509 relating to Probi® Fast

Melt technology, the entire contents of all three of which are incorporated herein by reference. Where the powder is not in a fast-melt microbial composition, it may be suitable for being added to a food (e.g. yoghurt) or drink (e.g. water or milk) before ingestion.

Where the composition is in the form of a powder, it would typically be filled in a sealed container, which provides an oxygen and moisture barrier in order to protect and maintain the viability of the probiotic bacteria in the composition. Hence, where the composition is in the form of a powder, preferably the composition is packaged in sealed aluminium foil sticks, where each stick comprises one dose of the composition, i.e. one dose of the probiotic bacteria. Non-limiting examples of suitable containers include a stick, bag, pouch or capsule. In a preferred embodiment, the container is an aluminium foil or a polyethylene stick, which is typically sealed by welding. The stick is typically configured for easy tear opening. The stick may have a tear notch.

The composition according to the first aspect of the invention may be formulated as a controlled-release solid dosage form, for example any of those described in WO 03/026687 and U.S. Pat. Nos. 8,007,777 and 8,540,980, the entire contents of which are incorporated herein by reference. The composition may be formulated as a layered dosage form, for example Probi's BIO-tract® technology including any of the layered dosage forms described in WO 2016/003870, the entire contents of which are incorporated herein by reference.

A tablet may be made by compression or moulding, optionally with one or more accessory ingredients. Compressed tablets may be prepared by compressing in a suitable machine the at least one probiotic strain (e.g. freeze-dried) in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide the desired release profile.

Pharmaceutical Compositions

A second aspect of the invention provides a pharmaceutical composition comprising the at least one probiotic strain according to the first aspect of the invention, and one or more pharmaceutically acceptable excipients, for use in the treatment and/or prevention of trabecular bone loss, in a mammal, preferably a non-rodent mammal, more preferably in a human, most preferably in a post-menopausal woman.

The pharmaceutical composition according to the second aspect of the invention may be a composition as described above in respect of the first aspect of the invention. The term “pharmaceutically acceptable” includes that the one or more excipients must not be deleterious to the recipients thereof and must be compatible with the at least one probiotic strain according to the first aspect of the invention. Examples of such pharmaceutically acceptable excipients are well known in the art and include those described above in respect of the first aspect of the invention, for example those described in Remington: The Science and Practice of Pharmacy, 19^{th} ed., vol. 1 & 2 (ed. Gennaro, 1995, Mack Publishing Company).

For example, the pharmaceutical composition may be formulated as a controlled-release solid dosage form, e.g. any of those described in WO 03/026687 and U.S. Pat. Nos. 8,007,777 and 8,540,980, or the pharmaceutical composition may be formulated as a layered dosage form, e.g. any of those described in WO 2016/003870.

The one or more pharmaceutically acceptable excipients may be water or saline which will be sterile and pyrogen free.

Preferably, the pharmaceutical composition according to the second aspect of the invention may be administered by any conventional method including oral and tube feeding. Administration may consist of a single dose or a plurality of doses over a period of time.

Methods of Treatment

A third aspect of the invention provides a method for treating and/or preventing trabecular bone loss in a mammal, comprising administering to a mammal in need thereof a therapeutically effective amount of the at least one probiotic strain according to the first aspect of the invention, the composition according to the first aspect of the invention, or the pharmaceutical composition according to the second aspect of the invention.

In particular, the methods according to the third aspect of the invention include those wherein the prevention of trabecular bone loss is by reducing trabecular bone loss compared to not having been administered said probiotic strains.

The mammal on which the methods according to the third aspect of the invention are carried out may be any mammal given above in relation to the first aspect of the invention. For example, the mammal may be a man. The mammal may be a woman, such as a peri-menopausal woman or a post-menopausal woman. Preferably, the mammal is a woman within one, two, three, four, five, six, seven, eight, nine, ten, eleven or twelve years from onset of menopause, and most preferably a woman six years or less after onset of menopause. Hence, in one embodiment of the methods according to the third aspect of the invention, treatment commences six years or less after onset of menopause.

Administration according to the methods of the third aspect of the invention may include administration orally, buccally or sublingually as described above in relation to the first aspect of the invention.

Administration according to the methods of the third aspect of the invention may include administration at least every one, two, three, four, five, six or seven days, or at least one, two, three, four, five, six or seven times a week. Preferably administration takes place once daily.

Administration according to the methods of the third aspect of the invention may include administration that is repeated for up to one, two, three, four, five or six days, for up to one, two, three, four or five weeks, for up to one, two, three, four, five, six, seven, eight, nine, ten, eleven or twelve months, or for more than one, two or three years or longer. Preferably, administration is repeated for at least 7 days, such as for at least one week, two weeks, three weeks, more preferably for at least four weeks, one month, two months or three months, and even more preferably for at least six months, nine months or one year.

Administration according to the methods of the third aspect of the invention is preferably of a unit dosage of from about 1×10⁶ to about 1×10¹⁴ CFU/unit dose, preferably from about 1×10⁸ to about 1×10¹² CFU/unit dose, more preferably from about 1×10⁹ to about 1×10¹¹ CFU/unit dose, and most preferably about 1×10¹⁰ CFU/unit dose, in accordance with the first aspect of the invention. Administration according to the methods of the third aspect of the invention preferably results in an effective dose of from about 1×10⁶ to about 1×10¹⁴ CFU/unit dose, preferably from about 1×10⁸ to about 1×10¹² CFU/unit dose, more preferably from about 1×10⁹ to about 1×10¹¹ CFU/unit dose, and most preferably about 1×10¹⁰ CFU/unit dose. Preferably, each subject is administered one unit dose per day. Hence, administration according to the methods of the third aspect of the invention preferably results in a daily dose of from about 1×10⁶ to about 1×10¹⁴ CFU/day, preferably from about 1×10⁸ to about 1×10¹² CFU/day, more preferably from about 1×10⁹ to about 1×10¹¹ CFU/day, and most preferably about 1×10¹⁰ CFU/day.

It will be appreciated that a preferable daily dose may also be achieved by administration of more than one sub-dose, for example, by a twice daily administration of a unit dose comprising half of the preferable daily dose. Hence, the preferred ranges for the effective dose may also represent the preferred daily dosage to be achieved in whatever number of unit doses is practical.

The subject may be instructed to consume the therapeutically effective amount of the at least one probiotic strain according the first aspect of the invention or the pharmaceutical composition according to the second aspect of the invention, in combination with water, another aqueous solvent or a food product, e.g. yoghurt.

Use in Treatment and/or Prevention

A fourth aspect of the invention provides the use of a composition comprising the at least one probiotic strain according to the first aspect of the invention, the composition according to the first aspect of the invention, or the pharmaceutical composition according to the second aspect of the invention, in the treatment and/or prevention of trabecular bone loss in a mammal.

The listing or discussion of an apparently prior-published document in this specification should not necessarily be taken as an acknowledgement that the document is part of the state of the art or is common general knowledge.

The invention will now be described in more detail by reference to the following Examples and Figures.

### BRIEF DESCRIPTION OF THE FIGURES

FIG. 1 discloses the relative change (percentage change from baseline) in bone mineral density (BMD) at lumbar spine after the intervention for 12 months with either the probiotic product or placebo. *** represents a within-group change of p<0.001. The probiotic product significantly reduces bone mineral density loss at the lumbar spine compared to placebo.

FIG. 2 discloses the relative change (percentage change from baseline) in bone mineral content (BMC) at lumbar spine after the intervention for 12 months with either the probiotic product or placebo. * represents a within-group change of p<0.05. The probiotic product significantly reduces bone mineral content loss at the lumbar spine compared to placebo.

FIG. 3 discloses the relative change (percentage change from baseline) in bone mineral density (BMD) at lumbar spine after the intervention for 12 months with either the probiotic product or placebo, in the subgroup of participants with osteopenia at baseline. * represents a within-group change of p<0.05. The probiotic product significantly reduces bone mineral density loss at the lumbar spine compared to placebo in the subgroup of participants with osteopenia at baseline.

FIG. 4 discloses the relative change (percentage change from baseline) in bone mineral density (BMD) at lumbar spine after the intervention for 12 months with either the probiotic product or placebo, in the subgroup of healthy participants with normal T-score (T≥−1 at total hip and lumbar spine) at baseline. * represents a within-group change of p<0.05. Bone mineral density at the lumbar spine was significantly reduced with placebo but not with the probiotic product.

FIG. 5 discloses the relative change (percentage change from baseline) in bone mineral density (BMD) at lumbar spine after the intervention for 12 months with either the probiotic product or placebo, in the subgroup of participants that had been less than 6 years from the start of menopause at baseline. *** represents a within-group change of p<0.001. The probiotic product significantly reduces bone mineral density loss at the lumbar spine compared to placebo in the subgroup of participants that had been less than 6 years from the start of menopause at baseline.

FIG. 6 discloses the relative change (percentage change from baseline) in bone mineral density (BMD) at lumbar spine after the intervention for 12 months with either the probiotic product or placebo, stratified in relation to the time from onset of menopause. Black * represents a within-group change of p<0.05; yellow * represents a between-groups difference of p<0.05; red * represents a between-groups difference of p=0.055.

FIG. 7 discloses the project plan diagram for Experimental Example 2.

FIG. 8 discloses the effect of probiotics on the tibial epiphyseal trabecular bone (trabecular bone at the proximal epiphysis of the tibia). (A) Trabecular bone volume/total volume (BV/TV) of knee joints of male mice, as assessed by microCT of the tibial epiphysis. (B) Trabecular thickness (Tb.Th); (C) Trabecular separation (Tb.Sp); (D) Trabecular number (Tb.N); and (E) Trabecular pattern factor (Tb.Pf), from the same experiment. Values are mean±SEM from 11-13 mice per group. Significant differences between groups indicated by *=p<0.05.

FIG. 9 discloses the effect of probiotics on the femoral epiphyseal trabecular bone (trabecular bone at the distal epiphysis of the femur). (A) Trabecular bone volume/total volume (BV/TV) of DMM-operated and unoperated knee joints of male mice, as assessed by microCT of the femoral epiphysis. (B) Trabecular thickness (Tb.Th); (C) Trabecular separation (Tb.Sp); (D) Trabecular number (Tb.N); and (E) Trabecular pattern factor (Tb.Pf), from the same experiment. Values are mean±SEM from 10-13 mice per group. Significant differences between groups indicated by *=p<0.05.

Exemplary Dosage Forms

In addition to the formulations referenced above (and incorporated herein by reference), the following examples illustrate pharmaceutical formulations according to the invention.

### EXAMPLE A: TABLET

| Probiotic | 1 × 10⁹ CFU or | |
|---|---|---|
| strain(s) | preferably 1 × 10¹⁰ CFU | |
| Lactose | 200 | mg |
| Starch | 50 | mg |
| Polyvinylpyrrolidone | 5 | mg |
| Magnesium stearate | 4 | mg |

Tablets are prepared from the foregoing ingredients by wet granulation followed by compression.

### EXAMPLE B: TABLET FORMULATIONS

The following formulations A and B are prepared by wet granulation of the ingredients with a solution of povidone, followed by addition of magnesium stearate and compression.

Formulation A

| | | |
|---|---|---|
| (a) Probiotic strain(s) | 1 × 10⁹ CFU* | 1 × 10⁹ CFU* |
| (b) Lactose B.P. | 210 mg | 26 mg |
| (c) Povidone B.P. | 15 mg | 9 mg |
| (d) Sodium Starch Glycolate | 20 mg | 12 mg |
| (e) Magnesium Stearate | 5 mg | 3 mg |

(*= or preferably 1 × 10¹⁰ CFU)

Formulation B

| | | |
|---|---|---|
| (a) Probiotic strain(s) | 1 × 10⁹ CFU* | 1 × 10⁹ CFU* |
| (b) Lactose | 150 mg | — |
| (c) Avicel PH 101 ® | 60 mg | 26 mg |
| (d) Povidone B.P. | 15 mg | 9 mg |
| (e) Sodium Starch Glycolate | 20 mg | 12 mg |
| (f) Magnesium Stearate | 5 mg | 3 mg |

(*= or preferably 1 × 10¹⁰ CFU)

Formulation C

| | |
|---|---|
| Probiotic strain(s) | 1 × 10⁹ CFU or pre- |
| | ferably 1 × 10¹⁰ CFU |
| Lactose | 200 mg |
| Starch | 50 mg |
| Povidone | 5 mg |
| Magnesium stearate | 4 mg |

The following formulations, D and E, are prepared by direct compression of the admixed ingredients. The lactose used in formulation E is of the direction compression type.

Formulation D

| | |
|---|---|
| Probiotic strain(s) | 1 × 10⁹ CFU or pre- |
| | ferably 1 × 10¹⁰ CFU |
| Pregelatinised Starch NF15 | 150 mg |

Formulation E

| | |
|---|---|
| Probiotic strain(s) | 1 × 10⁹ CFU or preferably 1 × 10¹⁰ CFU |
| Lactose | 150 mg |
| Avicel ® | 100 mg |

Formulation F (Controlled Release Formulation)

The formulation is prepared by wet granulation of the ingredients (below) with a solution of povidone followed by the addition of magnesium stearate and compression.

| | |
|---|---|
| (a) Probiotic strain(s) | 1 × 10⁹ CFU or preferably 1 × 10¹⁰ CFU |
| (b) Hydroxypropylmethylcellulose | 112 mg |
| (Methocel K4M Premium) ® | |
| (c) Lactose B.P. | 53 mg |
| (d) Povidone B.P.C. | 28 mg |
| (e) Magnesium Stearate | 7 mg |

Release takes place over a period of about 6-8 hours and was complete after 12 hours.

### EXAMPLE C: CAPSULE FORMULATIONS

Formulation A

A capsule formulation is prepared by admixing the ingredients of Formulation D in Example B above and filling into a two-part hard gelatin capsule. Formulation B (infra) is prepared in a similar manner.

Formulation B

| | |
|---|---|
| (a) Probiotic strain(s) | 1 × 10⁹ CFU or preferably 1 × 10¹⁰ CFU |
| (b) Lactose B.P. | 143 mg |
| (c) Sodium Starch Glycolate | 25 mg |
| (d) Magnesium Stearate | 2 mg |

Formulation C

| | |
|---|---|
| (a) Probiotic strain(s) | 1 × 10⁹ CFU or preferably 1 × 10¹⁰ CFU |
| (b) Macrogol 4000 BP | 350 mg |

Capsules are prepared by melting the Macrogol 4000 BP, dispersing the probiotic strain(s) in the melt and filling the melt into a two-part hard gelatin capsule.

Formulation D (Controlled Release Capsule)

The following controlled release capsule formulation is prepared by extruding ingredients a, b, and c using an extruder, followed by spheronisation of the extrudate and drying.

The dried pellets are then coated with release-controlling membrane (d) and filled into a two-piece, hard gelatin capsule.

| (a) Probiotic strain(s) | 1 × 10¹⁰ CFU or pre- | |
|---|---|---|
| | ferably 1 × 10¹⁰ CFU | |
| (b) Microcrystalline | 125 | mg |
| Cellulose | | |
| (c) Lactose BP | 125 | mg |
| (d) Ethyl Cellulose | 13 | mg |

### EXAMPLE D: POWDER FORMULATIONS

Formulation A (Fast-Melting Microbial Composition)

| (a) Probiotic strain(s) | 80 | mg |
|---|---|---|
| | (preferably 1 × | |
| | 10¹⁰ CFU) | |
| (b) Erythritol | 450 | mg |
| (c) Inulin | 227.5 | mg |
| (d) Xylitol | 227.5 | mg |
| (e) Lemon flavour | 10 | mg |
| (f) Silicon dioxide | 5 | mg |

Formulation B (Fast-Melting Microbial Composition)

| (a) Probiotic | 80 | mg |
|---|---|---|
| strain(s) | (preferably 1 × | |
| | 10¹⁰ CFU) | |
| (b) Erythritol | 425 | mg |
| (c) Inulin | 215 | mg |
| (d) Xylitol | 215 | mg |
| (e) Maltodextrin | 50 | mg |
| (f) Lemon flavour | 10 | mg |
| (g) Silicon dioxide | 5 | mg |

### EXPERIMENTAL EXAMPLE 1

Materials and Methods

Study Design

This was a multicenter, randomized, double-blind, placebo-controlled clinical study including 249 healthy women in early post-menopausal phase. The randomization into one of the two study groups, probiotics or placebo, was done using a computerized random number generator. All study participants were assigned a screening number from the beginning and once they were found eligible for participation in the study they were randomly allocated a randomization number. Written informed consent was obtained from all participants before screening for eligibility and before enrolment in the study. The primary endpoint was to study the percent change in BMD at lumbar spine, based on dual energy x-ray absorptiometry (DXA), after 12 months of intervention with either the probiotic product or placebo.

Inclusion and Exclusion Criteria

The following inclusion criteria were applied: Healthy women in early post-menopausal phase (at least two years and a maximum of 12 years since the last menstruation and at least one year since the last intake of hormone replacement therapy); BMI≥18 and ≤30 at screening; BMD T-score in the lumbar spine (L1-L4)>−2.5, as measured by DXA; commitment not to use any products that may influence the study outcome in the opinion of the Investigator and ability to understand and comply with the requirements of the study, as judged by the Investigator. Participation in the study was not approved if any of the following exclusion criteria was fulfilled: Relevant history of >1 previous fracture after 50 years of age, as judged by the Investigator; T-score≤−2.5, in the total hip or lumbar spine (L1-L4). These subjects should be forwarded to a GP for further investigation; history of metabolic bone disease; unstable weight (±five [5] kg) during the last six (6) months; history of hyperthyroidism or unstable hypothyroidism; diagnosed with disease causing secondary osteoporosis within the last year, including primary hyperparathyroidism, chronic obstructive pulmonary disease, inflammatory bowel disease (IBD), celiac disease or diabetes; known history of rheumatoid arthritis, clinically significant kidney or heart disease, as judged by the Investigator; gastric bypass surgery performed; history of immunodeficiency or immunosuppressive treatment; chronic or acute diarrheal disease; recently diagnosed malignancy (within the last five [5] years); use of products containing probiotic bacteria (more than once per week) within four (4) weeks prior to baseline; per-oral use of corticosteroids; use of calcium and/or vitamin D supplements within one (1) month prior to baseline; use of any anti-resorptive therapy, including e.g. systemic hormone replacement therapy, bisphosphonates (currently or during last 12 months); use of any bone-formation stimulating therapy (currently or during the last 12 months); use of antibiotics during the last two (2) months; frequent user of antibiotics (>2 courses during the last 12 months) due to inter-current infection episodes; smoking or use of nicotine-containing products (currently or during the last six [6] months); history of alcohol abuse, or excessive intake of alcohol, as judged by the Investigator.

Study Procedures

During a pre-screening phone call, the subjects were checked for compliance with the eligibility criteria (excluding the DXA criteria), based on a medical history questionnaire.

Eligible subjects were scheduled for a DXA scan and if eligibility for the study was confirmed they were booked to Visit 1 (baseline visit; randomization) within two weeks.

Additional visits were conducted one (1), three (3), six (6) and 12 months after Visit 1 and the study participants were contacted by phone two (2) and nine (9) months after Visit 1 to confirm that they were taking the study product as planned and to collect information on any AEs and concomitant medications, as well as use of other products containing probiotics.

Fasting blood and urinary samples for the analysis of bone turnover markers (beta form of C-terminal telopeptide, CTx; ratio of N-terminal telopeptide/creatinine, NTx/Cr; osteocalcin, OC; procollagen type I N-terminal propeptide, P1NP) were taken at baseline, 1, 3, 6 and 12 months. The samples were kept frozen at −80° C. until analysis.

Intervention

The active investigational product (IP) consisted of a combination of the three (3) probiotic bacterial strains Lactobacillus paracasei 8700:2 (DSM 13434), Lactobacillus plantarum Heal 9 (DSM 15312) and Lactobacillus plantarum Heal 19 (DSM 15313). The IP was supplied in capsules containing a powder with freeze-dried bacteria and maize starch used as filler. Each bacterial strain was equally represented in the total bacterial dose of 1×10¹⁰ CFU/capsule.

The placebo capsules were of identical appearance, taste and texture as the active IP with the exception that the probiotic powder was substituted with yeast peptone.

The participants were instructed to consume one capsule daily for the total length of the study that was 12 months.

Bone Mineral Density and Bone Mineral Content Measurements

Bone mineral density (BMD) and bone mineral content (BMC) at lumbar spine L1-L4 (LS) were measured by dual energy X-ray absorptiometry (DXA) at the beginning and at the end of the study (12 months). The equipment used was calibrated according to the manufacturer's instructions and central reading of all DXA measurements was applied in the study.

Determination of Sample Size

It was estimated that when measuring the % change from baseline in the BMD at lumbar spine by DXA, to have an 80% chance to see a statistically significant difference of 2 between the active group and the placebo, a sample size of 100 subjects/group was required. Considering a possible drop-out rate of 20% it was decided to randomize 250 subjects in total, allocated to receive active product of placebo at a ratio of 1:1.

Statistical Methods

Wilcoxon rank-sum test was used for the comparison between the groups whereas Wilcoxon signed rank test was applied for the analysis of the changes over time within the groups. Data presented in the results section correspond to the full analysis set (intention to treat) that consists of all subjects who were randomised into the study and received at least one (1) dose of the investigational product.

Results

Reduced Bone Loss at Lumbar Spine in the Probiotic Group

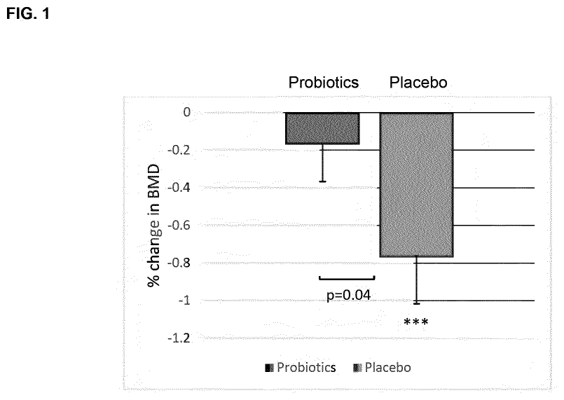

After the intervention period of 12 months, the LS-BMD was significantly reduced in the placebo group by 0.77% (p=0.0006) whereas in the probiotic group the reduction by 0.17% was not significant (p=0.40) (FIG. 1). Moreover, the loss in LS-BMD was significantly reduced in the probiotic group compared to placebo group (p=0.04; Wilcoxon rank-sum test).

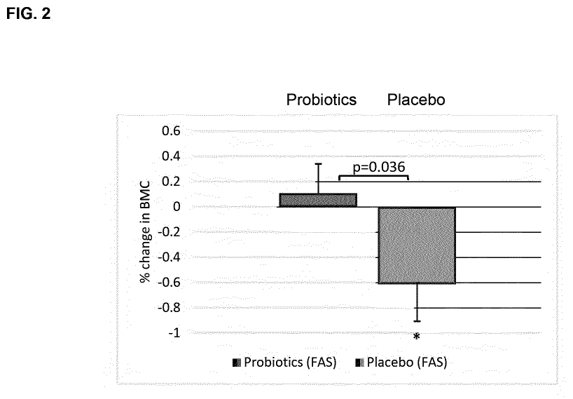

Looking at the changes in LS-BMC there was a significant mean reduction in the placebo group by 0.61% (p=0.018) whereas there was an increase in the mean relative change in the probiotic group by 0.1% (p=0.6). In accordance with the BMD and as shown in FIG. 2, intake of probiotics significantly counteracted the reduction of LS-BMC observed in the placebo group (p=0.036; Wilcoxon rank-sum test).

Higher Probiotic Efficacy in the Subgroup with Osteopenia

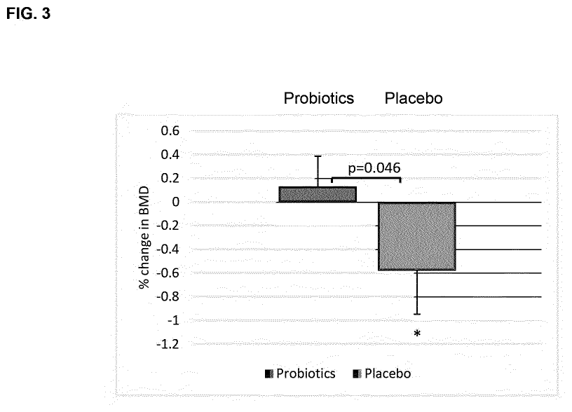

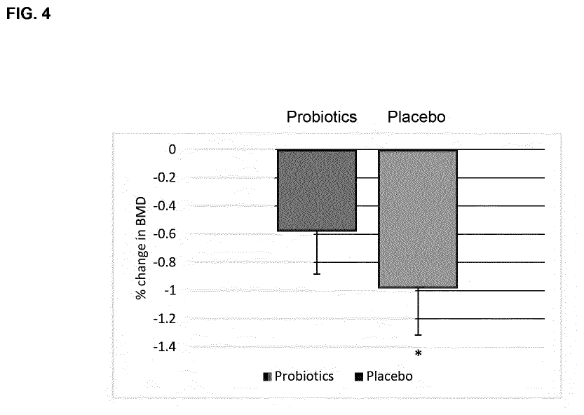

A subgroup analysis of the total population was conducted based on the baseline levels of T-score at total hip and spine. Study participants were described as healthy when the baseline T-score was ≥−1.0 and osteopenic when T-score was −2.5<T<−1.0. In both subgroups of osteopenic and healthy participants there was a significant reduction in LS-BMD in the placebo groups (p=0.046 and p=0.005 respectively) but not in the probiotic groups (FIG. 3 and FIG. 4). Moreover, there was a significant difference between the probiotic group and the placebo, in the subgroup of women with osteopenia (p=0.046; FIG. 3).

Association Between the Reduced Bone Loss in the Probiotic Group and the Time From Start of Menopause

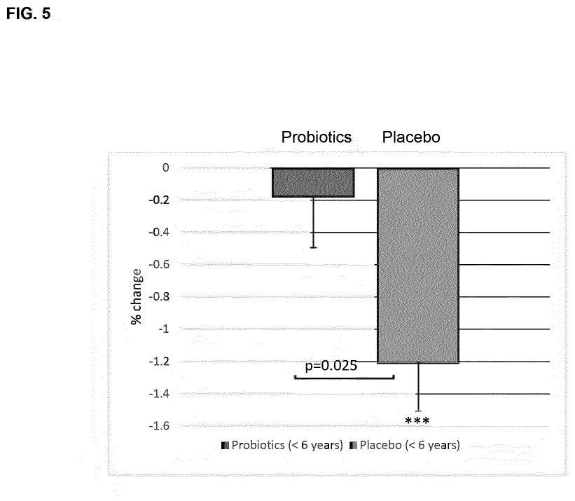

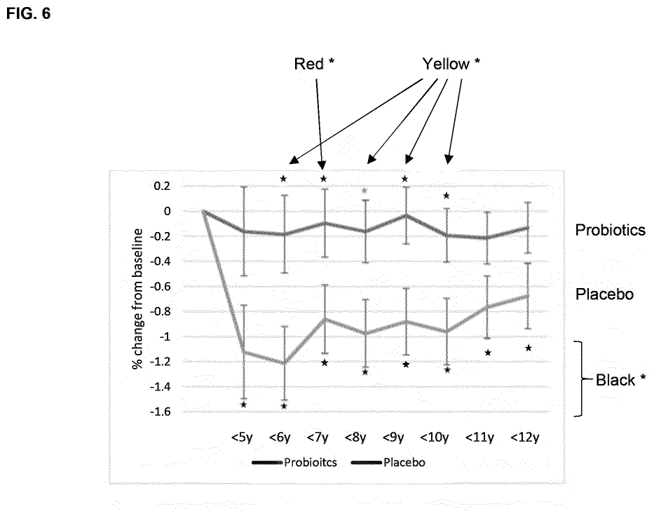

A second subgroup analysis was conducted based on the median time from the start of the menopause. The most pronounced protective effect of the probiotic treatment was observed in women below the median of 6 years from the start of menopause. For these early postmenopausal women, the LS-BMD loss was 0.18% in the probiotic treated group, a change that was significantly less (p=0.025) compared with the loss of 1.21% in the placebo group as shown in FIG. 5. The beneficial efficacy of the probiotic product on bone health at lumbar spine was clearly detectable at least 12 years after the start of menopause (FIG. 6), with the greatest difference between groups being at the time point of <6 years from menopause.

No Obvious Impact of the Probiotics on Markers for Bone Turnover

Serum and urine markers for bone turnover (CTx, NTx/Cr, OC, PN1P) were analyzed at different time points throughout the study. Although there were significant changes observed over time within each group there were no differences obtained between the probiotic group and placebo.

Conclusion

The overall conclusion from the results presented herein is that there is a clear beneficial effect from use of the probiotic product on bone health at lumbar spine in early postmenopausal women. The clearest benefit is observed in women with osteopenia that are expected to have a higher rate of bone resorption and associated inflammatory activity. Further subgroup analysis linked to the duration of women's menopause phase by the time they were recruited into the study shows that the protective effect of the probiotic product was clear across all subgroups (from less than 5 years from onset of menopause to at least less than 12 years from onset of menopause), with the most beneficial effect seen in the subgroup less than 6 years from onset of menopause. We believe the peak beneficial effect seen in the subgroup less than 6 years from onset of menopause may be due to a higher bone turnover and especially bone resorption activity during the first 5-6 years following the onset of the menopause compared to later years from onset of menopause.

### EXPERIMENTAL EXAMPLE 2

Materials and Methods

Study Design

Antibiotic treatment was used to deplete mouse intestinal microbes as previously shown (Ellekilde et al, 2014, Sci Rep 4:5922; Reikvam et al, 2011, PLoS One 6(3):e17996). Initially, ampicillin was administered to pregnant mice in drinking water ad libitum from one week before birth until the progeny mice reach the age of 3 weeks (i.e. age of weaning). In addition to ampicillin in drinking water, an antibiotic cocktail consisting of vancomycin, neomycin, metronidazole and amphotericin-B ampicillin was administered to the progeny mice daily by gavage as previously described (Reikvam et al, 2011, PLoS One 6(3):e17996) for 3 weeks after weaning. At the end of that period (six weeks after birth) faecal samples from healthy mice were aseptically removed and transplanted to the antibiotic-treated hosts (faecal microbiota transplantation, FMT), as previously described (Ellekilde et al, 2014, Sci Rep 4:5922). Briefly, samples were diluted 1:10 in a 50% glycerol/PBS solution, frozen in liquid nitrogen and kept in −80° C. At the day of the inoculation, the faecal solution was further diluted in 1:5 and then administered via oral gavage (0.15 ml per recipient mouse). This enabled the gut microbiome to be restored. Sham reconstitution of the gut microbiome involved oral gavage of glycerol/PBS solution.

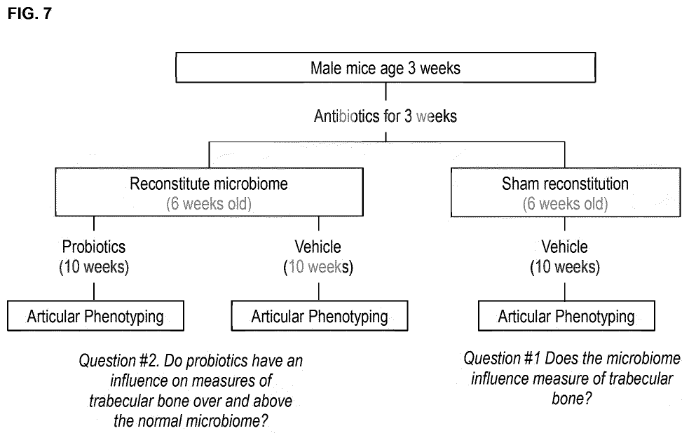

Probiotic or vehicle (glycerol) treatment began at the age of 6 weeks old and continued until the age of 16 weeks old. Probiotic treatment was a mixture of three Lactobacillus (L) strains, L. paracasei DSM 13434, L. plantarum DSM 15312 and DSM 15313. Lactobacillus strains were administered in drinking water according to instructions provided by Probi AB (10 mL of study product, containing equal amounts of each one of the three bacterial strains, was diluted with water to 600 mL in order to have a final total concentration of 10⁹ CFU/mL. Control groups received water with vehicle (glycerol) (See FIG. 7 for project plan diagram). At 16 weeks old, mice were sacrificed by CO₂ asphyxiation and knee joints were scanned by microCT to look for quantitative and qualitative changes in subchondral bone.

MicroCT analysis of knee joints was performed as described in van 't Hof (2012, Analysis of bone architecture in rodents using microcomputed tomography, Methods Mol Biol 816:461-476), Bouxsein et al (2010, Guidelines for assessment of bone microstructure in rodents using micro-computed tomography, J Bone Miner Res 25:1468-1486) and Campbell and Sophocleous (2014, Quantitative analysis of bone structure by micro-computed tomography, BoneKEy Reports, 3:564). Analysis of periarticular bone was performed by microCT as previously described in Sophocleous et al (2015, The type 2 cannabinoid receptor regulates susceptibility to osteoarthritis in mice, Osteoarthr Cartil 23:1585-1594). Briefly, a Skyscan 1172 instrument was set at 60 kV and 167mA. Tibial and femoral epiphyseal trabecular bone analysis was performed in the coronal plane, at a resolution of 5 mm. Following acquisition, the images were reconstructed using the Skyscan NRecon programme and analysed using Skyscan CTAn software.

MicroCT analysis was performed on the following three groups of male mice:
Group 1: “Sham FMT+glycerol” (mice that had no faecal microbiota transplantation and only administration of vehicle [glycerol]);
Group 2: “FMT+glycerol” (mice that had faecal microbiota transplantation followed by administration of vehicle [glycerol]);
Group 3: “FMT+probiotics” (mice that had faecal microbiota transplantation followed by administration of probiotics).

Bone mineral density (BMD) was not measured in this animal study, but trabecular bone volume (BV/TV) was measured instead.

Statistical Methods

Significant differences between groups were assessed using one-way analysis of variance (ANOVA) followed by Tukey HSD post hoc test (for equal variances) or Games—Howell post hoc test (for unequal variances). The significance level was set at p<0.05.

Results

Increased Measures of Tibial Epiphyseal Trabecular Bone at the Knee Joint with Probiotic Administration

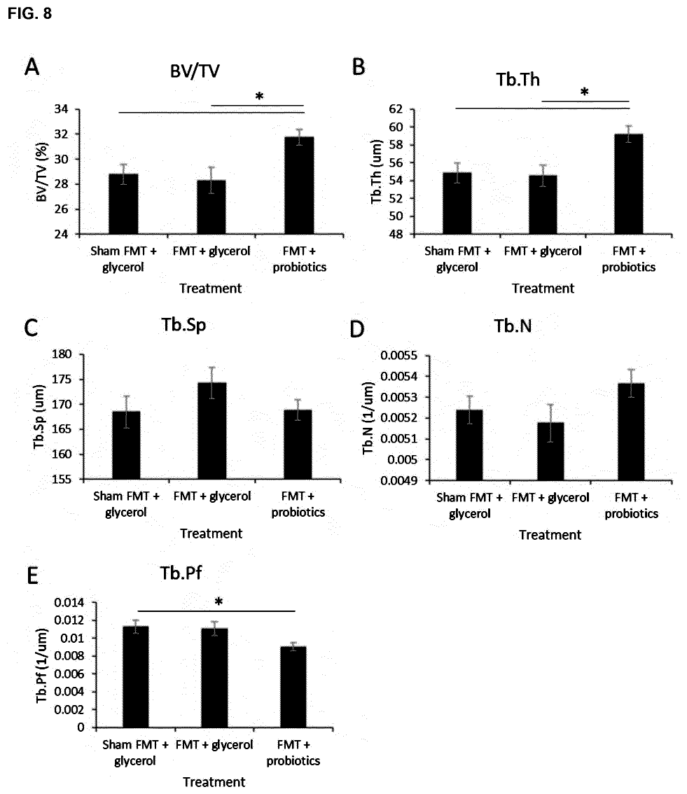

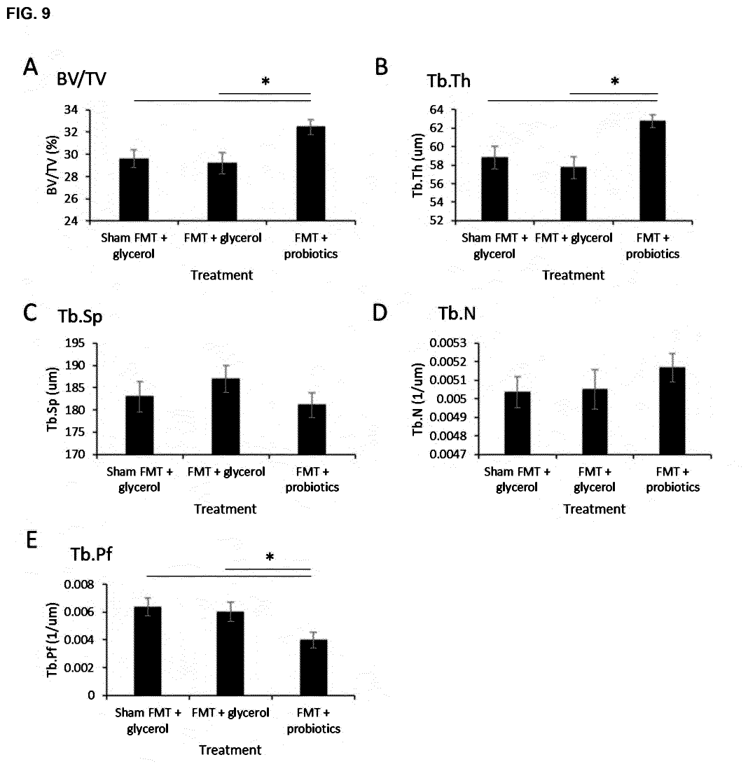

FIGS. 8 and 9 show that reconstituting the microbiome with FMT did not have a significant impact on any trabecular bone indices measured in this study.

FIG. 8A shows that tibial epiphyseal trabecular bone volume (as a proportion of total volume) was increased in the “FMT+probiotics” group compared to both the “FMT+glycerol” and “Sham FMT+glycerol” groups. Likewise, FIG. 8B shows that tibial epiphyseal trabecular thickness was increased in the “FMT+probiotics” group compared to both the “FMT+glycerol” and “Sham FMT+glycerol” groups.

FIG. 8C and FIG. 8D show that tibial epiphyseal trabecular separation and tibial epiphyseal trabecular number, respectively, were not significantly different between the groups.

However, FIG. 8E shows that tibial epiphyseal trabecular pattern factor was reduced in the “FMT+probiotics” group compared to the “Sham FMT+glycerol” group. Trabecular pattern factor (Tb.Pf) is related to trabecular connectivity. A higher Tb.Pf value indicates lower connectivity, while a lower Tb.Pf value indicates better (higher) connectivity amongst trabeculi. Hence, the results show that probiotic administration improved tibial epiphyseal trabecular connectivity. It appears likely that this result is due to increased trabecular thickness (FIG. 8B) and overall increased trabecular bone volume (FIG. 8A) rather than due to increased trabecular number (FIG. 8D).

Increased Measures of Femoral Epiphyseal Trabecular Bone at the Knee Joint with Probiotic Administration

Administration of probiotics (“FMT+probiotics” group) increased femoral epiphyseal trabecular bone volume (as a proportion of total volume; BV/TV; FIG. 9A) and femoral epiphyseal trabecular thickness (Tb.Th.; FIG. 9B) compared to both the “FMT+glycerol” and “Sham FMT+glycerol” groups.

FIG. 9C and FIG. 9D show that femoral epiphyseal trabecular separation and femoral epiphyseal trabecular number, respectively, were not significantly different between the groups.

FIG. 9E shows that femoral epiphyseal trabecular pattern factor was reduced in the “FMT+probiotics” group compared to both the “FMT+glycerol” and “Sham FMT+glycerol” groups, indicating that probiotic administration improved femoral epiphyseal trabecular connectivity. Like for tibial epiphyseal trabecular connectivity, it appears likely that this result is due to increased trabecular thickness (FIG. 4B) and overall increased trabecular bone volume (FIG. 4A) rather than due to increased trabecular number (FIG. 4D).

Conclusions

Hence, these data further support those of Experiment 1 in showing that administration of probiotic treatment (the combination of L. paracasei DSM 13434, L. plantarum DSM 15312 and DSM 15313) increases measures of trabecular bone, in this case at the knee joint. The effect was observed in both tibial and femoral epiphyseal bone.

1. A method for treating and/or preventing trabecular bone loss in a mammal, comprising administering to a mammal in need thereof an effective dose of at least one probiotic strain chosen from Lactobacillus paracasei 8700:2 (DSM 13434) and/or at least one probiotic strain of Lactobacillus plantarum.

2. The method according to claim 1, wherein the mammal is a human.

3. The method according to claim 2, wherein the human is a peri-menopausal woman or a post-menopausal woman.

4. The method according to claim 2, wherein the human is a woman six years or less after onset of menopause.

5. The method according to any one of claims 1-4, wherein the effective dose of the at least one probiotic strain is administered at least once a day or at least every two, three, four, five, six or seven days, or at least one, two, three, four, five, six or seven times a week.

6. The method according to any one of claims 1-5, wherein the effective dose of the at least one probiotic strain is from about 10⁶ to about 10¹⁴ colony forming units (CFU) per dose, preferably from about 10⁸ to about 10¹² CFU per dose, or more preferably from about 10⁹ to about 10¹¹ CFU per dose.

7. The method according to claim 6, wherein the effective dose of the at least one probiotic strain is about 10¹⁰ CFU per dose.

8. The method according to any one of claims 1-7, wherein one or more effective doses of the at least one probiotic strain are administered in one day, and wherein the daily dose of the at least one probiotic strain is from about 10⁶ to about 10¹⁴ CFU per day, preferably from about 10⁸ to about 10¹² CFU per day, or more preferably from about 10⁹ to about 10¹¹ CFU per day.

9. The method according to claim 8, wherein the daily dose of the at least one probiotic strain is about 10¹⁰ CFU per day.

10. The method according to any one of claims 1-9, wherein the treatment and/or prevention of trabecular bone loss in a mammal involves increasing the absorption of Ca²⁺ ions.

11. The method according to any one of claims 1-10, wherein the at least one probiotic strain of Lactobacillus plantarum is chosen from Lactobacillus plantarum 299 (DSM 6595), Lactobacillus plantarum 299v (DSM 9843), Lactobacillus plantarum HEAL 9 (DSM 15312), Lactobacillus plantarum HEAL 19 (DSM 15313), Lactobacillus plantarum HEAL 99 (DSM 15316), Lactobacillus plantarum GOS42 (DSM 32131), Lactobacillus plantarum DSM 17852 (LB3e) and Lactobacillus plantarum DSM 17853 (LB7c).

12. The method according to claim 11, wherein the at least one probiotic strain is Lactobacillus paracasei 8700:2 (DSM 13434) in combination with Lactobacillus plantarum HEAL 9 (DSM 15312) and Lactobacillus plantarum HEAL 19 (DSM 15313).

13. The method for use according to any one of claims 1-12, wherein the at least one probiotic strain is administered in a composition comprising at least one carrier selected from a pharmaceutically acceptable carrier, a pharmaceutically acceptable excipient, a food-grade carrier, a food-grade excipient, a diluent, and a food.

14. The method according to claim 13, wherein the composition is provided in the form of a solution, suspension, emulsion, tablet, granule, powder, capsule, lozenge, chewing gum, or suppository.

15. The method according to claim 13, wherein the food is a cereal-based product, a dairy product, a juice drink, or a fermented food.

16. The method according to any one of claims 13-15, wherein the composition is supplemented with vitamin D, for example selected from vitamin D3 cholecalciferol or vitamin D2 ergocalciferol.

17. At least one probiotic strain chosen from Lactobacillus paracasei 8700:2 (DSM 13434) and/or at least one probiotic strain of Lactobacillus plantarum, for use in the treatment and/or prevention of trabecular bone loss in a mammal.

18. A composition comprising the at least one probiotic strain according to claim 17, and one or more carriers, for use in the treatment and/or prevention of trabecular bone loss in a mammal.

19. A pharmaceutical composition comprising the at least one probiotic strain according to claim 17, and one or more pharmaceutically acceptable excipients, for use in the treatment and/or prevention of trabecular bone loss in a mammal.

20. Use of a composition comprising the at least one probiotic strain according to claim 17, or use of a composition or pharmaceutical composition according to claim 18 or 19, in the treatment and/or prevention of trabecular bone loss in a mammal.
